(19) United States
(12) Patent Application Publication
(10) Pub. No.: US 2016/0073953 A1
(43) Pub. Date: Mar. 17, 2016

(54) FOOD INTAKE MONITOR

(71) Applicant: Board of Trustees of The University of Alabama, Tuscaloosa, AL (US)

(72) Inventor: Edward Sazonov, Northport, AL (US)

(21) Appl. No.: 14/845,855

(22) Filed: Sep. 4, 2015

Related U.S. Application Data
(60) Provisional application No. 62/049,001, filed on Sep. 11, 2014.

(51) Int. Cl. A61B 5/00; A61B 5/11

(52) U.S. Cl. CPC A61B 5/4866; A61B 5/4542; A61B 5/1114; A61B 5/7282; A61B 5/0024; A61B 5/7267; A61B 5/486; A61B 5/742; A61B 5/6801; A61B 2019/5214

(57) ABSTRACT

Systems and methods for monitoring food intake include, in one exemplary embodiment, a jaw sensor configured to detect jaw motion and an accelerometer configured to body motion. The system may also include, for example, a hand gesture sensor configured to detect a hand motion and a central processing unit configured to determine whether the jaw motion, the body motion, and the hand motion are associated with food intake.

### CROSS REFERENCE TO RELATED APPLICATIONS

This application claims priority to U.S. Provisional Patent Application No. 62/049,001 entitled “Food Intake Monitor” and filed Sep. 11, 2014, the content of which is herein incorporated by reference in its entirety.

### BACKGROUND

The prevalence of obesity in developed countries is increasing at an alarming rate. Obesity contributes to an increased risk of heart disease, hypertension, diabetes, and some cancers and is now considered a risk factor for cardiovascular disease. Millions of people are attempting to lose weight at any time, but the rate of success at preventing weight regain remains low.

The research community devotes a significant effort toward studying effects of energy intake and expenditure on energy balance and weight gain. A fundamental baseline for each person measures how much consumed food and associated calories are required for effective weight loss or gain. Various techniques have been used to record food intake, including keeping a personal record or using a software application on a personal computer, PDA or smartphone. These techniques, however, rely on a user to record or take pictures of every meal and the portions received, which proves unlikely in practice. Other techniques have sought to automatically monitor food intake. For example, a wearable system may listen for the sound of a person swallowing or chewing to determine the rate of food consumption or count the number of hand-to-mouth gestures (“bites”). Even these wearable systems, however, either too imprecise (such as sound-based approaches) or require input from a user (such as the hand gesture counters). A user must turn the gesture counter on or off when consuming a meal to avoid the possibility of falsely recording consumption of food throughout the day.

At the present time there is no accurate, inexpensive, non-intrusive way to objectively quantify energy intake in free living conditions and study behavioral patterns of food consumption.

### SUMMARY

Systems and methods for monitoring food intake include, in one exemplary embodiment, a jaw sensor configured to detect jaw motion and an inertial measurement unit configured to measure body and/or head motion. The system may also include, for example, a hand gesture sensor configured to detect a hand motion and/or hand proximity to the mouth and a central processing unit configured to determine whether the jaw motion, the body motion, and/or the hand motion are associated with food intake. The system may also include a camera which is triggered by detected food intake to take pictures of the foods being eaten. The system may also include software to characterize food intake in terms of duration, rate of ingestion, calories and nutrients consumed from the automatic or manual analysis of the sensor signals and/or food imagery.

### BRIEF DESCRIPTION OF THE DRAWINGS

FIGS. 1A-1D illustrates exemplary components for monitoring ingestive behavior.

FIG. 2A illustrates an exemplary system for monitoring ingestive behavior.

FIG. 2B illustrates a second exemplary system for monitoring ingestive behavior.

FIG. 3 illustrates an exemplary flowchart for signal processing.

FIG. 4 illustrates a second exemplary flowchart for signal processing.

FIG. 5 illustrates an exemplary graph for monitoring food intake.

FIG. 6 illustrates a third exemplary flowchart for signal processing.

FIG. 7 illustrates an exemplary flowchart for monitoring food intake.

FIG. 8 illustrates examples of implementing the feedback algorithms on AIM data.

FIG. 9 illustrates an exemplary processing system consistent with various embodiments.

### DESCRIPTION

The disclosed systems and methods provide an automated wearable device for monitoring ingestive behavior, caloric and nutrient intake and optionally modifying ingestive behavior and caloric intake using real-time feedback from the wearable system. A human can be considered a thermal and mass exchange system. The underlying physical principle is the law of conservation of energy and matter.

Conservation of mass under normal conditions over some considerably long period T can be expressed by the following formula taking into account major components:

$${\int }_{T} ( {M}_{FOOD} + {M}_{{O}_{2}} ) = {\int }_{T} ( {M}_{WEIGHTGAIN} + {M}_{{CO}_{2}} + {M}_{Fecal} + {M}_{Urinary} + {M}_{Evaporation} ) \qquad ( 1 )$$

Conservation of energy under normal conditions (constant body temperature) for a period T can be expressed by the following formula:

$${\int }_{T} {E}_{FOOD} = {\int }_{T} ( {E}_{MECHWORK} + {E}_{HEAT} + {E}_{STORAGE} + {E}_{WASTE} ) , \qquad ( 2 )$$

where E_{FOOD} is energy content of food intake (digestible chemical energy+heat energy), E_{MECHWORK} is energy spent for external work (force x distance), E_{HEAT} is energy lost as heat, E_{STORAGE} is energy stored in protein, carbohydrate and fat storage, and E_{WASTE} is chemical energy of food, which was not consumed and lost through excretions.

The systems and methods disclosed herein provide techniques to quantify energy and nutrient intake. Most of the energy intake in humans comes from food. By monitoring chewing (mastication) and swallowing (deglutition), food intake quantities can be estimated. In one exemplary embodiment, deglutition (swallowing) can be reliably identified by a device detecting characteristic sounds in the area lateral or caudal to the laryngeal prominence. Deglutition can also be identified by a device detecting characteristic sounds in the mastoid bone, detecting electrical impulses resulting from muscle activation during swallowing or by detecting changes in electrical impedance of the laryngeal region during swallowing. Mastication (chewing) creates specific motion of the lower jaw that can be identified by a device detecting motion of the mandible and/or skin in the region of the outer ear. Mastication can also be identified by a device detecting characteristic sound in the mastoid bone, ear canal or detecting deformation inside of the ear canal, as well as by detecting electrical signals resulting from muscle activation during jaw motion.

Wearable non-intrusive sensors may detect deglutition through a sound sensor located in the area caudal to the laryngeal prominence. Another exemplary embodiment may detect deglutition through a behind-the-ear sound sensor and detect mastication through a behind-the-ear strain sensor. Alternatively, optical, tactile or magnetic sensors may be used located at various locations around the body. Further, signal processing methods and pattern recognition methods may automatically detect deglutition and mastication. A classification algorithm may utilize signals from mastication and/or deglutition sensors as predictors and identify periods of food consumption, recognize and identify individual foods in the meal, or trigger a camera that captures the image of the food being eaten.

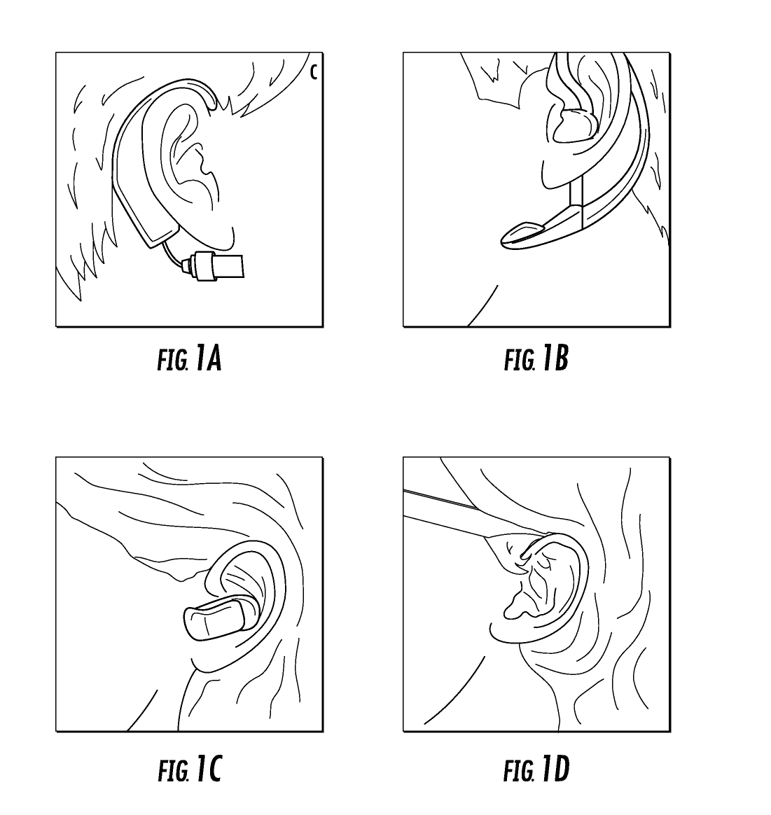

FIG. 1A illustrates a first exemplary embodiment that includes a piezoelectric sensor that may be worn in the area immediately below the outer ear. The sensor may detect changes in the skin curvature created by the characteristic motion of the mandible during chewing of food. In one exemplary embodiment, a buffered signal from the sensor may be acquired by a data acquisition system.

FIGS. 1B and 1C illustrate exemplary embodiments that relies on a sound, strain, tactile, optical or magnetic sensor in a boom of a headset worn over the ear and detecting jaw motion or/and sensor in the ear canal detecting chewing sound or deformation of the ear canal. FIG. 1D illustrates another exemplary embodiment with the sensors integrated into the frames of eye glasses either directly in front of the ear (straight or curved temples) or behind the ear (temple tips that may be elongated to reach lower ear).

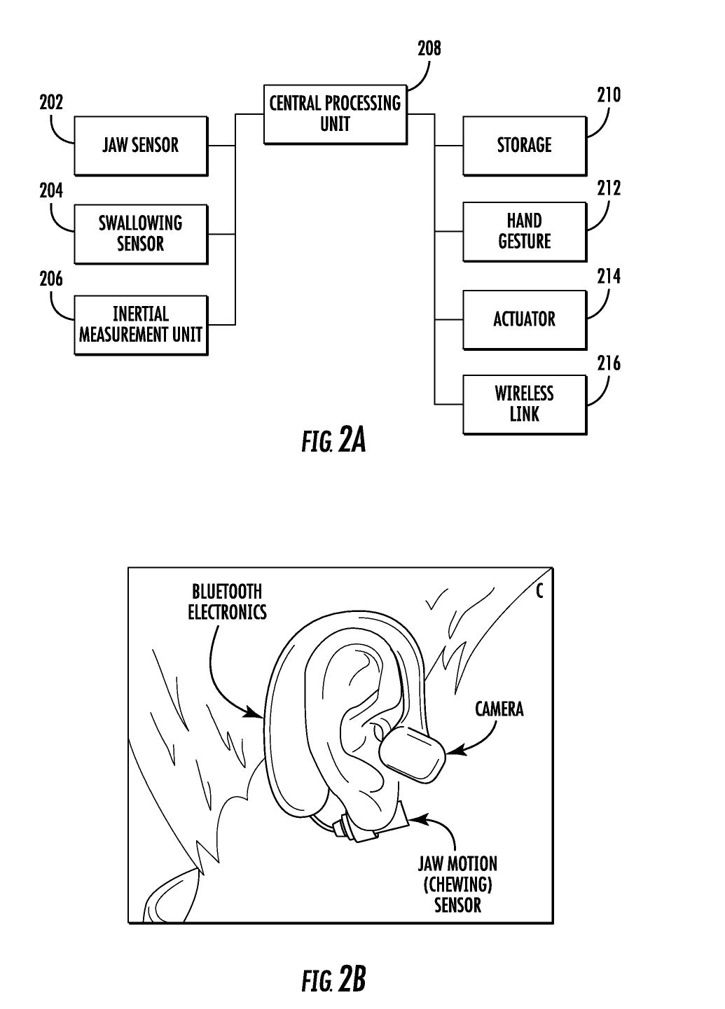

FIG. 2A illustrates a system including a jaw sensor 202, such as the piezoelectric sensor, strain sensor, magnetic or optical sensor, a swallowing sensor 204 that detects deglutition by monitoring sounds, mechanical motion, electrical potentials or electrical signals in the laryngeal area, an inertial measurement unit 206 that detects body motion, a hand gesture sensor 212 that detects hand motion and/or proximity of the hand to the mouth, an actuator 214 (for example, a vibrator or an ear phone) that delivers real-time feedback to the user, and an external wireless link 216 (such as Wi-Fi or Bluetooth) that delivers sensors data and/or food information or imagery to a remote server such as smart phone, personal or cloud computer. The system need not contain all of the devices 202, 204, 206, 212, 214 and 216, as the system may be configured with fewer than all four sensors, without the internal actuator or wireless link to an external device. For example, the jaw sensor 202, inertial measurement unit 206 and/or hand gesture sensor 212 may combine to detect food consumption, without swallowing sensor 204, or may be used individually to detect food consumption. Central processing unit 208 may perform signal processing to detect food consumption and store signals and historical trending data in storage 210. The items in FIG. 2 may be connected using an internal wireless link, and one or more of the items may be combined into a single component.

The swallowing sensor 204 may be a microphone specific for this application or one typically used for hands-free radio communications. It may also be a mechanical sensor, such as an accelerometer or strain sensor that detects displacement of the laryngopharynx in the absolute or relative to the inertial measurement unit's 206 frame of reference. It may also be an electrical electrode sensor that detects electrical potentials on the surface of the neck resulting from muscle excitation during swallowing. It may also be an electrical impedance sensor where a small DC or AC current is injected into the transmission electrode and received on the receiver electrode to detect swallowing and passing of the food bolus through the laryngopharynx. The waveform may be digitized by a sound card and a sound recording application at the sampling rate of, for example, 8000 Hz, although other sampling rates may be used. The swallowing sensor 204 may be positioned around the neck or on the mastoid bone behind the ear. The swallowing sensor 204 can be worn as a medallion attached, on a neck band or as a self-adhesive strip, offering a non-intrusive, wearable device that does not need special attention. A swallowing sound has a unique time-frequency pattern that can be identified by pattern recognition methods. Temporary medical adhesives may be used for providing better contact between the sensor and surface of the skin, or utilize an in-ear probe.

In one embodiment, jaw sensor 202 may be a piezoelectric, foil or ink-printed strain sensor that detects the specific motion of the lower jaw by capturing strains created by motion of the posterior border of the mandible's ramus, deformations on the surface of the skin during chewing, or vibrations propagated through the tissues during food crushing while chewing. Such a sensor may be attached to the skin or reside in an enclosure such as the boom in FIG. 1b or FIG. 1c without attachment to the skin but remaining in contact with the skin. In another embodiment, jaw sensor 202 may be an optical or magnetic sensor that detects skin surface deformation and/or motion during chewing without direct contact with the skin or body tissues. Such a sensor may or may not need additional optical or magnetic markers placed on the skin below the sensor. In another embodiment, jaw sensor 202 may be a tactile sensor that detects skin motion or vibrations from skin that is in the contact but free to slide under the sensor. In another embodiment, jaw sensor 202 may be electrical electrodes that detect electrical potentials from jaw muscle actuation during chewing.

These two sensors can be integrated into a single device worn behind the ear in a manner similar to a wireless phone headset, such as an earpiece or in frames of eye glasses. No special fittings or positioning of the sensors are required. Further, the sensors may be disguised as or integrated into a headset for a cellular phone.

The inertial measurement unit 206 may contain a micro-electromechanical, piezoelectric or other type of accelerometer and/or gyroscope and/or magnetometer. The inertial measurement unit may be sensitive to 1 to 9 dimensions of measurements such as linear acceleration, angular velocity or magnetic field.

The hand gesture sensor 212 may detect proximity to the mouth by using an RF strength measurement between a transmitter located on one of the user's arms and a receiver located in the headset or frames of the glasses; or detect the motion of bringing one's hand to the mouth through the means of inertial measurement unit that is placed on an arm, such as a wrist unit or a unit integrated into clothing. The hand gesture sensor may also detect hand proximity through passive capacitive coupling with the hand, or coupling from AC potentials injected at the device location. Several of the hand gesture detection methods can be combined to increase reliability of detection or to minimize number of wearable pieces.

Various types of microphones may be used as swallowing sensors 204. For example, a piezoelectric bone-conduction microphone may be used with high dynamic range and low power consumption. The sensor may be modified to be placed on the mastoid bone behind the ear or used as an ear probe. As another example, a piezoelectric noise-canceling microphone may be used, which has relatively small dimensions, a high dynamic range, and low power consumption. A third exemplary model may be a modified throat microphone usually used for hands-free radio communications. The throat microphone may be designed to pick up vibration signals from the surface of the skin, rather than waves of sound pressure. As such it may be highly insensitive to external noise, but sensitive to low-level sounds providing a dynamic range of, for example, 58 db and low power consumption of, for example, 0.5 ma at 3V. The microphone may be worn on an elastic band around the neck.

An exemplary jaw sensor may include a film piezoelectric sensor encased in a thin strip of elastic polymer material that regains shape after being deformed. The sensor may detect mastication (chewing) by identifying specific motion of the lower jaw. Essentially, it detects changes in skin curvature created by motion of the posterior border of the mandible's ramus. The surface of the sensor may be, in one embodiment, polished or treated with a low friction material to avoid abrasion. The sensor may be attached to the skin by an adhesive or held in contact with the skin by applying mechanical force from the wearable device. Exemplary embodiments include a behind-the-ear module or both behind-the-ear and laryngeal modules being worn by a subject.

Data from the sensors may continuously transmit to the pocket or wrist storage unit that accumulates data in memory, such as on a Secure Digital (SD) card. The storage unit may be a separate component or included in a personal computer, cell phone, smart phone, watch, or the like.

In some embodiments, a hand gesture sensor 212 may be added to identify the hand-to-mouth motion associated with eating. The timing and duration of food intake instances may be measured and monitored along with the number of bites, chews and swallows. A wireless module may include one or more of an accelerometer to capture body acceleration and may be integrated into, for example, a watch. A push button may also optionally be included to self-report food intake by the user to initially calibrate the system to a particular individual.

Examples of signal processing by central processing unit 208 consistent with one or more embodiments will now be described. The signal processing may be done locally by a processor integrated into the wearable device, a processor on a handheld device (e.g., a cell phone), a remote server or a combination of these configured in such manner as to extend the battery life of the wearable device. Initial signal processing of swallowing sensor data may include preamplification and low pass or band pass filtering with cutoff frequency of, for example, 3500 Hz. Preamplification cascade may allow for impedance matching and amplification of weak signals from the sensor before subjecting them to any further processing. The peak frequency detected by the swallowing sensor varies individually with subject and food type. In one example, the low pass filtering with a cutoff at 3500 Hz may be used to pass the spectrum of a swallow sound, while rejecting excessive high frequency noise and preventing aliasing during analog-to-digital conversion.

The dynamic range of signal from the swallowing sensor may be in the range of, for example, 40-60 dB, which may be insufficient to reliably capture the signals originating from swallowing without saturating the amplification circuits during normal speech. Therefore, the signal from the sensor may be preamplifed by an Automatic Gain Control (AGC) amplifier.

The signal from the output of the variable gain amplifier may be sampled by, for example, a Successive-Approximation-Register (SAR) analog-to-digital converted at the sampling frequency of 10000 Hz, which provides accurate sampling of high frequency components in the filtered sensor signal and avoids aliasing. The preamplification coefficient can be scaled up to 40 dB by a variable-gain amplifier, giving average resolution of about 18-19 effective bits. Additional signal processing may be employed to enhance resolution.

The sampled signal may be compressed using lossless and fast adaptive Huffman coding and transmitted to the pocket/wrist module, a cell phone, storage 210, or any other device. Gain values for automatic gain control may be stored along with the sampled analog signal to serve as a predictor. In addition or alternatively, the sample signals may be communicated wirelessly, such as through a WiFi or Bluetooth connection, to a nearby electronic device.

Central processing unit 208 may take the signal from the jaw motion sensor and low pass filter it with a cut-off frequency, such as 30 Hz-300 Hz. The signal may be sampled by an analog-to-digital converter at the sampling rate of, for example, 1000 Hz. The sensor data may also be transmitted to the portable device (e.g. smartphone) or remote server and used in pattern recognition of mastication, or processed directly on the wearable device.

FIG. 2B illustrates another embodiment with a camera may be integrated into the system to take images of food. The camera may be used in addition to, or instead of, any of the sensors discussed with reference to FIG. 2A previously. Adding a camera may allow more precise calculation of the type of food being consumed and reduce or eliminate false positives in food intake detection by allowing for visual validation of each intake episode. The direction of the camera's optical axis should follow the natural line of gaze, as it is typical to look at the foods being eaten during the picking up or biting the foods. The camera can be integrated into the over-the-ear or behind the ear headset, or inside of a glasses frame.

To save battery power the camera is kept in powered down mode and only is turned on for brief moments of time to take pictures every 1-100 s. Internal temporary image buffer keeps the history of several previous images to accommodate for the fact that the image has to be taken prior to detected jaw motion due to food intake. Once food intake is detected, the appropriate image is taken out of temporary memory buffer and saved or transmitted for processing. Image capture can also be triggered by hand gestures. The relative timing of the food intake detection events and frequency and timing of the image capture can be probabilistically optimized to maximize the likelihood of capturing the foods while minimizing camera's power consumption. The goal is to minimize frequency with which images are taken and keep the camera in low-power (sleep) state while maximizing probability of capturing a clear image of the food being eaten.

The camera may utilize the inertial measurement unit 206 to take clear pictures. The inertial measurement unit may be used to identify moments of least head motion and capture still images without motion blur. The images may be retaken if the inertial sensor readings suggest possibility of a blurred image. The inertial measurement unit may also be used to estimate field of vision during a meal and capture images covering the full scene. The head motion during a typical meal or snack has a limited and well-defined range of motion covering the full scene containing the foods. Use of inertial measurements will allow reconstruction of the relative location of the camera's optical axis and capture of images covering the whole field of view and recovering the scene at the analysis stage.

Additional image processing from the camera may include image filtering, scene reconstruction from partially occluded images and depth estimation from camera motion. The captured images may contain images of low quality, or images of the items not related to the foods being eaten. Since a redundant number of images is captured, such images may be discarded by filtering algorithms. Images captured by the wearable camera may also be partially occluded and contain various views of the scene. Automatic computer algorithms may use the still image sequence and inertial sensor readings to reconstruct the full scene and recover distances to the objects and object sizes.

Using imagery allows, automatically, without any participation from the wearer, images to be captured, stored, and wirelessly transmitted when the food intake is detected, thus capturing composition and energy density of the food. A nutritionist or an automatic computer algorithm may use these images to obtain energy density and portion size estimates. The energy density and portion size estimates with or without swallowing, chewing and hand gesture based estimates of ingested mass can then be used to estimate the energy consumed at each snack and meal.

The images may be used to identify foods and determine portion size based on container, plate, and cup sizes. The information may be automatically entered into tracking software and use a reference database containing the total energy, macro- and micro-nutrient content of all USDA food items. The outcome of image analysis will be numeric estimates of mass (M_{IMG}), energy content (EC_{IMG}) and energy density (ED_{IMG}) for each food item. Total energy intake may be computed as EI=ΣE^{N}ᵢ₌₁EDⁱ_{IMG}(Mⁱ_{IMG}+Mⁱ_{HG+CH})/2, where N is the number of food items.

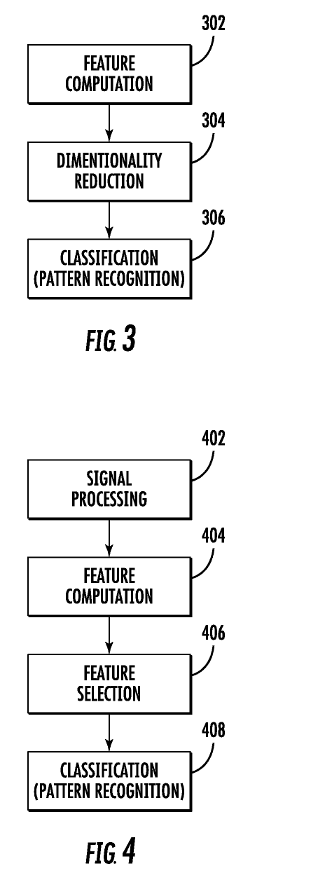

FIG. 3 illustrates an exemplary flowchart for signal processing of swallowing sensor signal after initial signal processing. One example of pattern recognition for deglutition may utilize a time-frequency decomposition method, such as Short-Time Fourier Transform (STFT) for feature extraction, Principal Component Analysis (PCA) for reduction of dimensionality and Multi-Layer Perceptron (MLP) artificial neural network for classification. In this example, the signal may be split into short intervals (epochs) with the size in the range of, for example, 50 to 30000 ms. Duration of an epoch may determine the balance between frequency and temporal resolution of the swallowing signal analysis.

At step 302, feature computation including the short-time Fourier Transform may be calculated for each epoch:

$${X}_{m} ( k ) = {\sum }_{n = 0}^{N - 1} w ( n ) x ( n + mN ) {e}^{- j {w}_{k} n} ,$$

where Xₘ(k) is the SFTF for the epoch m; N is the size of an epoch in samples; w(n)=0.5+0.5 cos(2πn/N) is the Hanning windowing function to reduce spectral leakage. Next, k Power Spectral Density coefficients are calculated for each epoch. Together with the optional AGC gain value, they may form the initial predictor vector v. The number of elements in v may also be reduced by a dimensionality reduction method (step 304) such as PCA, forming a reduced predictor v′.

The pattern recognition (classification) step 306 may use a MLP neural network with vectors v′ᵢ, (i=1, m) as inputs. The MLP accepts the input vector, propagates the vector through the its artificial neurons and produces a label of ‘0’ or ‘1’ on its output. The label indicates whether the epoch in question contains a swallow or not. The classification label is then passed for further processing that is used to detect and characterize food intake from chewing and/or swallowing and hand gesture sequences. The MLP network may be implemented using floating point or fixed point precision arithmetic, with the use of former targeting power savings on processors without hardware acceleration for floating point operations. The MLP network has to be trained prior to its use. Training of the network can be performed on the “gold standard” data collected from a population of individuals (performed once during the design stage) and further adapted to individual patterns using self-report data.

The training of the MLP may follow the Levenberg-Marquadt or other algorithms. Training may be performed once on a dataset collected from a population, thus resulting in a neural network classifier that does not need individual calibration before use. The MLP network may also be trained from data collected on a given individual, thus resulting in individually-calibrated recognition model.

A second exemplary pattern recognition technique for deglutition may use a discretized version of Continuous Wavelet Transform (CWT) for feature extraction, PCA for reduction of dimensionality, and Dynamic Time Warping (DTW) with nearest neighbor classification. To extract features, a discretized version of CWT algorithm may be used on epochs.

$${CWT}_{x}^{ψ} ( τ , s ) = {Ψ}_{x}^{ψ} ( τ , s ) = \frac{1}{\sqrt{\vert s \vert}} {\sum }_{t = 1}^{N} x ( t ) {ψ}^{*} ( \frac{t - τ}{s} ) ,$$

where τ represents translation, s represents scale, and Ψ*(t) is the mother wavelet. A Morlet mother wavelet may be used, which is defined as Ψ*(t)=e^{jat}e^{-t}^{²}^{/2s}, where a is a modulation parameter. Wavelet coefficients and optional AGC gain form the initial vector predictor vectors vₘ. Principal component analysis may be applied in the same manner as for the SFTF/MLP method and reduced-dimensionality feature vectors v′ₘ are formed.

Classification of the swallowing sounds may follow the Dynamic Time Warping technique. In one example, the classification scheme is built around N (10−1000) clear recordings of the swallowing sound that serves as the perfect class instances. A fuzzy expert system may use gain, amplitude and duration of signals to roughly identify potential swallows on the recordings. The DTW procedure may be applied to the test regions on the recording to compare them to the reference sounds and establish the measure D(X,Rᵢ)=min ∀φΣ^{T}ₖ₌₁d(φₓ(k),φ_{R}_{ᵢ}(k))f(k)/M_{φ}, where X is the test sound, Rᵢ is the i^{th} reference sound, φ is a warping path, T is the path length, d is the distance measure between features of X and Rᵢ, f is the slope weight, M_{φ} is the global path weight.

The result of DTW procedure is N metrics D(X, Rᵢ), establishing how close the test sound is to the reference sounds. These metrics classify the test sound as a swallow sound if min ∀i D(X, Rᵢ)<e, where e is the experimentally determined detection threshold.

Pattern recognition of swallowing may also employ machine learning techniques tailored to minimization of power consumption in the wearable device such as decision trees, random forests, logistic discrimination, Bayesian networks and other techniques that present relative light computational load to the processor. The pattern recognition may be split between the processor of the wearable device, performing first level detection with potentially high level of false positives at a low computational (and energy cost) and storing/wirelessly transmitting such epochs for more computationally intensive, but more accurate processing on the smart phone or in the cloud.

A set of useful characteristic such as number of swallows, swallowing frequency, variation of the swallowing sequence in time may be useful to analyze ingestive behavior of a person (potentially in combination with chewing metrics and hand gesture metrics): detect periods of food intake, identify solid and liquid intake, detect number of unique foods in a meal, and estimate mass and caloric intake.

The pattern recognition technique for detection of mastication may operate on the time series data acquired by the jaw motion sensor and be based on the fact that masticatory movements are characteristically periodical.

FIG. 4 illustrates another exemplary embodiment for detection of mastication. At step 402, the signal from the jaw motion sensor may be band-pass filtered to remove high-frequency noise and low-frequency drift of the zero axis. At step 404, a feature vector fᵢ∈ [symbol] ^{d} representing each epoch (for i=1, 2, . . . , N; where N is the total number of epochs) may be created by combining a set of 25 scalar features extracted from the filtered and unfiltered signal of each epoch in linear and logarithmic scale. This set of 25 features may include time domain and frequency domain features shown in Table I.

TABLE 1
SCALAR FEATURES USED TO EXTRACT
INFORMATION FROM CHEWING SIGNAL

| eat # | Description |
|---|---|
| 1 | RMS |
| 2 | Entropy (signal randomness) |
| 3 | Base 2 logarithm |
| 4 | Mean |
| 5 | Max |
| 6 | Median |
| 7 | Max to RMS ratio |
| 8 | RMS to Mean ratio |
| 9 | Number of zero crossings |
| 10 | Mean time between crossings |
| 11 | Max. time between crossings |
| 12 | Median time between cross. |
| 13 | Minimal time between cross. |
| 14 | Std. dev. of time between crossings |
| 15 | Entropy of zero crossings |
| 16 | Number of peaks |
| 17 | Entropy of peaks |
| 18 | Mean time between peaks |
| 19 | Std. dev. of time between peaks |
| 20 | Ratio peaks/zero crossings number |
| 21 | Ratio zero crossings/peaks number |
| 22 | Entropy of spectrum |
| 23 | Std. dev. of spectrum |
| 24 | Peak frequency |
| 25 | Fractal dimension (uniqueness of the elements inside an epoch) |

An initial feature vector fᵢ may be created merging several feature subsets that were formed by calculating the 25 scalar features form the filtered and unfiltered epoch and by different feature combinations:

fᵢ={f_{filt}, f_{unfilt}, f_{filt/unfilt}, f_{unfilt/filt}, f_{filt·unfilt}} (3)

where f_{filt} and f_{unfilt} represent feature subsets extracted from the filtered and unfiltered epochs respectively; f_{filt/unfilt} and f_{unfilt/flit} represent two feature subsets obtained by calculating the ratio between each feature of the f_{filt} and f_{unfilt} subsets and vice versa; and f_{filt·unfilt} represents another subset of features obtained by calculating the product between each feature of the f_{filt} and f_{unfilt} subsets. These combinations yield an initial feature vector with 125 dimensions.

A scale equalization may be performed to features in the f_{filt} and f_{unfilt} subsets using the natural logarithm. Ratio and product between resulting feature subsets may be calculated to create a log-scaled feature vector with 125 dimensions:

f_{log i}={f_{log filt}, f_{log unfilt}, f_{log filt/log unfilt}, f_{log unfilt/log filt}, f_{log filt·log unfilt}} (4)

Finally, both the linear and log-scaled feature vectors may be concatenated into a single 250-dimension feature vector Fᵢ∈ [symbol] ²⁵⁰ representing each epoch

Fᵢ={fᵢ, f_{log i}} (5)

To account for the time-varying structure of the chewing process, features from neighboring epochs may be added to the original epoch feature vector according to the number of lags selected L. Different lag values may be applied: 0-10. If the number of lags is greater than zero, then features from L previous and L subsequent epochs are included in the final feature vector τᵢ:

τᵢ={F_{i-L}, . . . , Fᵢ₋₁, . . . , Fᵢ, Fᵢ₊₁, . . . , F_{i+L}} (6)

At step 406, most important features may be selected using forward feature selection procedure or other feature selection algorithm. Features that contribute the most to detection of mastication are identified (selected) at this step. Step 406 may only be needed during the initial training of the algorithm for detection of mastication. In one example, features that were selected in step 406 may be computed in step 404 after training, thus saving the power required for computation.

At step 408, the feature vectors are processed by a pattern recognition algorithm (classifier) such as Support Vector Machine, Artificial Neural Network, Decision tree, Random Forest or other. The classifier may be trained on population data to enable detection of mastication without individual calibration, or trained on individual data to provide individual-specific recognition model. A combination of these may also be used, with initial model being trained on population data and further refined on individual data. The outcome of pattern recognition is that each recognized instance of deglutition and mastication are clearly identified by a binary label (0 or 1) on a timeline. A set of useful characteristic such as duration of mastication, number of chews, and chewing rate measured over recognized mastication sequences may be useful to analyze ingestive behavior of a person.

In another embodiment, a classification algorithm may use signals from mastication and/or deglutition sensors as predictors and identify periods of food consumption. The pattern recognition stage may be approached by means of a statistical method of logistic regression. The logistic regression provides not only common statistics (such as p-value) but also gives values of significance for each of the predictors and therefore indicates the relative importance of observing mastication or deglutition to characterize food consumption. Other benefits of logistic regression include small sample size to approximate normality and the fact that it cannot predict outside of the actual probability.

Logistic regression may be performed on two predictors xₘ and x_{d} which denote duration of mastication and frequency of deglutition within a time window of fixed length T respectively. Instead of assuming linear model on the response variable Yᵢ=βXᵢ+εᵢ in logistic regression it is applied to so “logit” function. That is:

ln

$$\frac{{p}_{i}}{1 - {p}_{i}} = β {x}_{i} + {ɛ}_{i} ,$$

or logit pᵢ=βxᵢ+εᵢ, where βXᵢ is a linear part with regular notation of components, i.e. β=(β₀,β₁, . . . , βₖ) denotes a vector of coefficients and xᵢ=(1,xᵢ₁,xᵢ₂, . . . , xᵢₖ) denotes a vector of data values, and pᵢ is P(Yᵢ=1). The model may then be designed to predict probability that the central point of the current window indicates food consumption, i.e. Y=1. The above formula is equivalent to

$$P ( {Y}_{i} = 1 \vert {x}_{i} ) = {p}_{i} = \frac{{e}^{β {x}_{i} + {ɛ}_{i}}}{1 + {ɛ}^{β {x}_{i} + {ɛ}_{i}}} .$$

To find optimal set of coefficients β the likelihood function

$$L ( β ) = {\prod }_{i = 1}^{N} {{p}_{i}^{{Y}_{i}} ( 1 - {p}_{i} )}^{1 - {Y}_{i}}$$

may be maximized. The conditions to solving this maximization problem can be translated into the following set of equations obtained by differentiating the above equation with respect to β:

$${\sum }_{i = 1}^{N} [ {Y}_{i} - p ( {x}_{i} ) ] = 0$$

$$and$$

$${\sum }_{i = 1}^{N} {x}_{ij} [ {Y}_{i} - p ( {x}_{i} ) ] = 0 , \\ j = 1 , 2 , … , p .$$

The following model provides an exemplary description of the prediction of the probability of food consumption p at central point with predictors specified above: Logit p=β₀+β₁xₘ+β₂x_{d}+ε.

Several measures are applied to evaluate the goodness-of-fit, predictive power and the significance of the model. The quality of the model as a whole is represented by the difference between the null and residual deviances: G_{M}=D₀−D_{M}. The test of significance of G_{M} (which under the null hypothesis is G_{M}˜χₖ²) is essentially the test of: H₀: β₁=β₂=β₃= . . . =βₖ=0 versus H₁: at least one of β is not equal to 0. The p-value, which is the probability that the large test statistic (G_{M}) has occurred due to a chance, i.e. p-value=P(χₖ²>G_{M}|H₀), can be obtained by using most statistical packages. The test of significance for any particular βⱼ employs Wald test statistics which is under the null hypothesis H₀: βⱼ=0 follows standard normal distribution:

$${W}_{j} = \frac{{b}_{j}}{S \hat{E} ( {b}_{j} )} ,$$

where SÊ(bⱼ)=[Vâr(bⱼ)]^{1/2}. The variances and covariances of estimated coefficients are obtained from the inverse of this matrix Var(β)=I^{-I}(β), where I(β) is observed information matrix, calculated as partial derivatives matrix of second order of the log-likelihood function.

In another embodiment, methods that are computationally simpler than logistic discrimination can used to detect food intake based on the detection of mastication and deglutition. For example, presence of mastication can be used as an indicator of food intake or frequency of deglutition exceeding the baseline (spontaneous swallowing frequency) by a certain proportion may be used to detect food intake. Other machine learning techniques such as decision trees, random forests or others can be used to detect food intake as well.

The detection of food intake is complicated by the fact that activities of a free living individual are complex and unpredictable. The sensor signals may be affected by activities other than food intake and therefore be confused for intake. For example, steps taken during walking may result in acoustical signals similar to those of swallowing sounds and therefore be confused for swallowing. To alleviate the problem and to increase reliability of food intake detection, the device may employ other sensors that help in differentiating food intake from other activities. Such sensors may include the hand gesture sensor, the inertial measurement unit and others. The information provided by these sensors may be used as stand-alone (e.g. number of hand gestures is indicative of ingested volume) or in combination with jaw motion and swallowing sensors (sensor fusion).

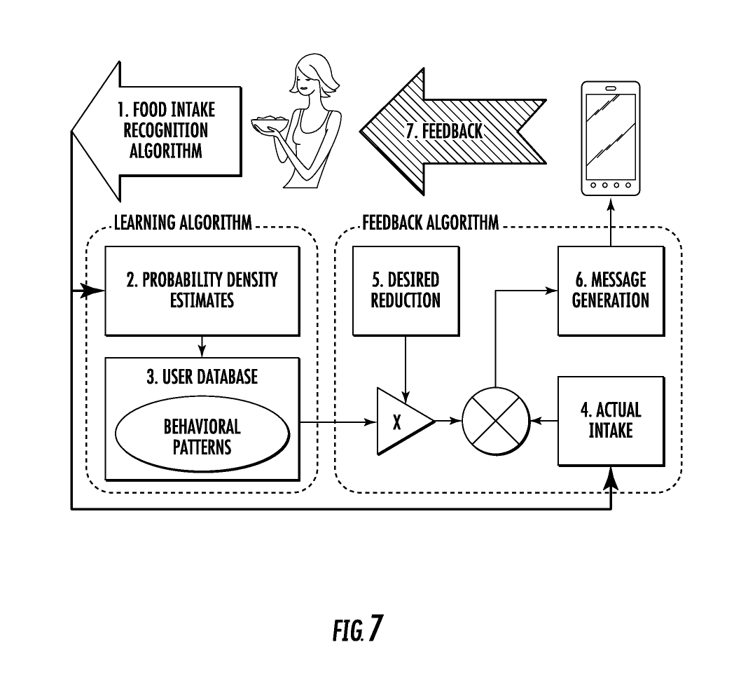

FIG. 6 illustrates an exemplary processing algorithm for detecting food intake by performing sensor fusion of jaw motion, hand gesture and inertial measurement unit signals.

The hand gesture sensor 212 on FIG. 2 may record signals indicating a gesture of bringing food to the mouth. The hand gesture senor may detect such gestures by measuring proximity of the hand to one's mouth by the means of RF or capacitive sensor, identifying orientation of the wrist in Earth's gravity field by the means of inertial measurement unit located on the wrist, detecting motion trajectory of the wrist during the gesture or combination of these measures. The outcome of hand gesture detection is an analog or digital signal HG(t) indicating hand-to mouth gestures.

The signal HG(t) may be fused with the jaw sensor signal JM(t) indicating jaw motion. The fusion may be performed to increase reliability of food intake detection and accurately differentiate food intake from other activities of daily living. The product between the absolute values of JM(t) and HG(t) may be computed at step 602 as: SF₁(t)=|JM(t)|·|HG(t)|. SF₁(t) may be divided into non-overlapping epochs eᵢ of 30 s duration with i=1, 2, . . . , M_{S} total number of epochs for each subject S. The size selected for the epoch may present the best trade-off between the frequency of physiological events such as bites, chewing and swallowing and time resolution of food intake monitoring. The Mean Absolute Value (MAV) of the signal SF₁/(t) within eᵢ may be computed as:

$${MAV}_{{e}_{i}} = \frac{1}{N} {\sum }_{k = 1}^{N} \vert {x}_{k} \vert$$

xₖ is the k-th sample in an epoch eᵢ of SF₁/(t) containing a total of N samples. The self-report signal, PB(t), may also be divided into 30 s epochs and used to assign a class label cᵢ∈{‘food intake’ (FI), ‘no food intake’ (NFI)} to each eᵢ during training of the sensor fusion algorithm to determine the rejection threshold T₁. The self-report signal may not be needed during normal operation of the food detection algorithm, but only used to collect data for training of the algorithms. An epoch may be labeled as food intake if at least 10 s of self-report within the i-th epoch was marked as food intake; otherwise it was labeled as not food intake. Other durations than ten seconds may be chosen.

SF₁(t) epochs would have higher MAV during food intake due to the presence of hand-to-mouth gestures (associated with bites and use of napkins) and jaw motion activity (chewing) during eating. For that reason, a threshold level T₁ may be set to remove epochs in SF₁/(t) belonging to activities that do not present a combination of jaw motion and hand gestures (i.e. sleeping, sitting quietly, working on a computer, watching TV, etc.).

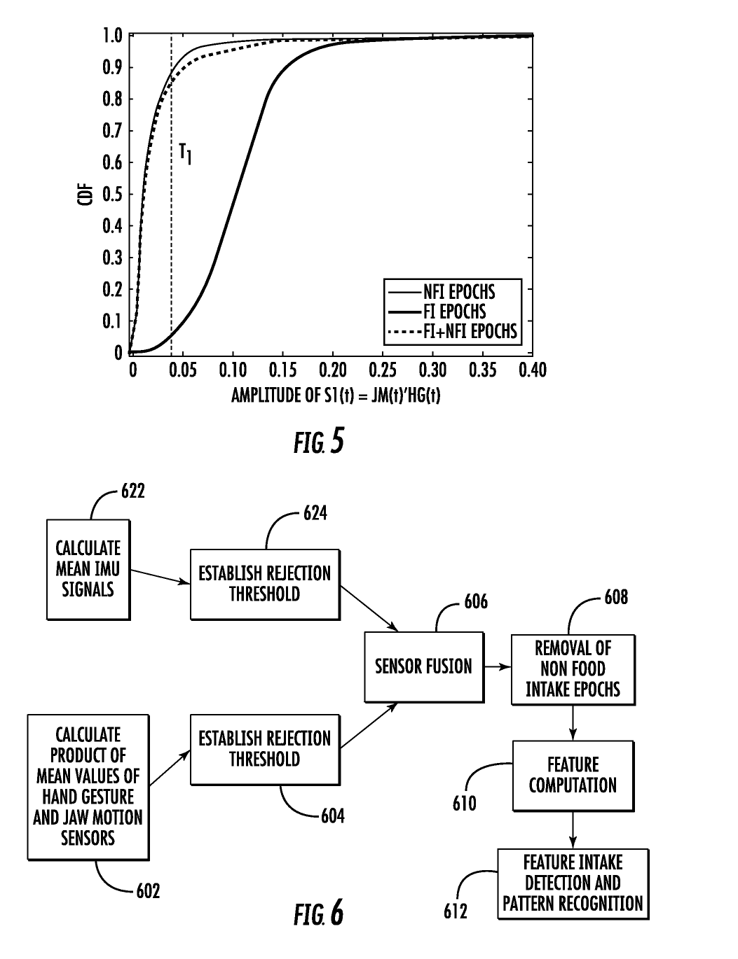

FIG. 5 illustrates the cumulative distribution function (CDF) of the MAV for food intake and not food intake epochs in SF₁/(t) for one subject. The CDF represents the probability that an epoch will have a MAV less than or equal to a certain number in the x-axis. The CDF for not food intake epochs grows faster than the CDF for food intake epochs, meaning that there is a high probability to find a not food intake epoch with low MAV but a low probability to find a food intake epoch with the same MAV and vice versa. A common threshold value, T₁, may be determined from the population data at step 604 and the indexes of the i-th epochs having a MAV below T₁ may be stored in a vector Idx_{SF1} indicating epochs that are not likely to be food intake. Determination of the threshold value T₁ from the population data may only be necessary during algorithm development and the established value of T₁ may be then used for anyone without a need for individual calibration or population value of T₁ may be used as initial estimate of threshold and then further adjusted from individual data.

Inertial measurement unit 206 on FIG. 2 may detect body motion signals. Data from sensor 206 can be used to identify when an individual is asleep to avoid recording false positives during rest. Further, individuals typically do not eat during rigorous exercise. Therefore, false positives associated with jaw motion and hand gesture signals while an individual breathes heavily and jogs, for example, can be avoided by measuring body acceleration to indicate ongoing exercise.

At step 622, the mean of the signals from the inertial measurement unit (such as 3-dimensional accelerations ACC_{X}(t), ACC_{Y}(t) and ACC_{Z}(t)) may be computed as:

SF₂(t)=⅓·(|ACC_{X}(t)|+|ACC_{Y}(t)|+|ACC_{Z}(t)|)

SF₂(t) may be divided into M_{S} non-overlapping epochs of 30 s duration and a class label cᵢ may be assigned to each epoch eᵢ as in the algorithm for processing of hand gesture signal. Since most of the individuals consume foods in a sedentary position, SF₂(t) epochs have higher MAV during activities involving body motion (i.e. walking, running, etc.) than during food intake. Thus, a common threshold value T₂ may be found for all subjects in the dataset at step 624 and the indexes of the i-th epochs in SF₂(t) with a MAV above T₂ may be stored in a vector Idx_{SF2} for further processing. Determination of the threshold value T₂ from the population data may only be necessary during algorithm development and the established value of T₂ may be then used for anyone without a need for individual calibration or population value of T₂ may be used as initial estimate of threshold and then further adjusted from individual data.

At step 606, sensor fusion may be performed by grouping a new vector Idx_{SF}={Idx_{SF1}∪Idx_{SF2}}∈ [symbol] ^{D}^{ₛ} with D_{S} <M_{S} total number of epochs for each subject S. Finally, at step 608 the signals JM(t), HG(t), ACCₓ(t), ACC_{y}(t),), ACC_{z}(t), and PB(t) for each subject may be divided into M_{S} non-overlapping epochs of 30 s duration, which were synchronized in time with SF₁(t) and SF₂(t) epochs. Thus, the epoch indexes stored in Idx_{SF} were used to label the sensor signals epochs as non-food intake and remove them from the dataset used in the pattern recognition task. As a result, a total of D_{S} epochs may be removed from the initial M_{S} epochs as non-food intake epochs. The remaining epochs need to be processed by feature computation and pattern recognition steps to identify food intake epochs.

Time and frequency domain features may be extracted at step 610 from the remaining epochs of the sensor signals and combined to create a feature vector fᵢ∈ [symbol] ⁶⁸ that represents an interval, such as 30 s. Each vector fᵢ may be formed by combining features from sensor signals as: fᵢ={f_{JM}, f_{HG}, f_{ACC}}, where f_{JM}∈ [symbol] ³⁸, f_{HG}∈ [symbol] ⁹, and f_{ACC}∈ [symbol] ²¹ represented the subsets of features extracted from JM(t), HG(t), and the inertial measurement unit (such as ACC(t)) signals respectively.

The subset f_{JM} may include time and frequency domain features extracted from each epoch of the jaw motion signal, as shown in Table II below). Frequency domain features may be computed from different ranges of the frequency spectrum of JM(t) within each epoch. The subset f_{HG} may include time domain features extracted from the hand-to-mouth gestures observed within each epoch (Table III).

The subset f_{ACC} contained time domain features computed from the accelerometer signals from each axis (Table IV). Features may include MAV, SD and the median value of the signal as well as number of zero crossings, mean time between crossings and entropy of the signal within the epoch. The means of the MAV, SD and entropy across the 3 axes may be computed to obtain a total 21 features.

TABLE II
FEATURES EXTRACTED FROM THE JAW MOTION SIGNAL

| # | Description |
|---|---|
| 1 | Mean Absolute Value (MAV) |
| 2 | Root Mean Square (RMS) |
| 3 | Maximum value (Max) |
| 4 | Median value (Med) |
| 5 | Ratio: MAV/RMS |
| 6 | Ratio: Max/RMS |
| 7 | Ratio: MAV/Max |
| 8 | Ratio: Med/RMS |
| 9 | Signal entropy (Entr) |
| 10 | Number of zero crossings (ZC) |
| 11 | Mean time between ZC |
| 12 | Number of peaks (NP) |
| 13 | Average range |
| 14 | Mean time between peaks |
| 15 | Ratio: NP/ZC |
| 16 | Ratio: ZC/NP |
| 17 | Wavelength |
| 18 | Number of slope sign changes |
| 19 | Energy of the entire frequency spectrum¹ (spectr_ene) |
| 20 | Energy spectrum in chewing range² (chew_ene) |
| 21 | Entropy of spectrum chewing range (chew_entr) |
| 22 | Ratio: chew_ene/spectr_ene |
| 23 | Energy spectrum in walking range³ (walk_ene) |
| 24 | Entropy of spectrum walking range (walk_entr) |
| 25 | Ratio: walk_ene/spectr_ene |
| 26 | Energy spectrum in talking range⁴ (talk_ene) |
| 27 | Entropy of spectrum talking range (talk_entr) |
| 28 | Ratio: talk_ene/spectr_ene |
| 29 | Ratio: chew_ene/walk_ene |
| 30 | Ratio: chew_entr/walk_entr |
| 31 | Ratio: chew_ene/talk_ene |
| 32 | Ratio: chew_entr/talk_entr |
| 33 | Ratio: walk_ene/talk_ene |
| 34 | Ratio: walk_entr/talk_entr |
| 35 | Fractal dimension |
| 36 | Peak frequency in chewing range (maxf_chew) |
| 37 | Peak frequency in walking range (maxf_walk) |
| 38 | Peak frequency in talking range (maxf_talk) |

¹Frequency range: 0.1-500 Hz;
²Chewing range: 1.25-2.5 Hz;
³Walking range: 2.5-10 Hz;
⁴Talking range: 100-300 Hz.

TABLE III
FEATURES EXTRACTED FROM
THE HAND GESTURE SIGNAL

| # | Description |
|---|---|
| 1 | Num. of HtM gestures within epoch (num_HtM) |
| 2 | Duration of HtM (D_HtM) |
| 3 | MAV of HtM |
| 4 | Stardard Deviation of HtM |
| 5 | Maximum value (Max_HtM) |
| 6 | Wavelength (WL) |
| 7 | Ratio: WL/Duration HtM |
| 8 | Ratio: D_HtM/num_HtM |
| 9 | Ratio: MAV_HtM/D_HtM |

TABLE IV
FEATURES EXTRACTED FROM
THE ACCELEROMETER SIGNALS

| # | Description |
|---|---|
| 1 | MAV of ACC_{X} (MAVx) |
| 2 | SD of ACC_{X} (SDx) |
| 3 | Median of ACCy |
| 4 | Num. of zero crossings (ZC) for ACCx |
| 5 | Mean time between ZC for ACCx |
| 6 | Entropy of ACCx (Entrₓ) |
| 7 | MAV of ACCy (MAVy) |
| 8 | SD of ACCy (SDy) |
| 9 | Median of ACCy |
| 10 | Num. of zero crossings for ACCy |
| 11 | Mean time between ZC for ACCy |
| 12 | Entropy of ACCy (Entr_{y}) |
| 13 | MAV of ACCz (MAVz) |
| 14 | SD of ACCz (SDz) |
| 15 | Median of ACCz |
| 16 | Num. of ZC for ACCz |
| 17 | Mean time between ZC for ACCz |
| 18 | Entropy of ACCz (Entr_{z}) |
| 19 | Mean of {MAVx, MAVy, MAVz} |
| 20 | Mean of {SDx, SDy, SDz} |
| 21 | Mean of {Entrₓ, Entr_{y}, Entr_{z}} |

Finally, each feature vector fᵢ may be associated with a class label tᵢ∈{1,−1}, where tᵢ=1 and tᵢ=−1 represented food intake and not food intake, respectively. The same rule used in the Sensor Fusion step was used here to assign class labels to each fᵢ vector. A dataset containing the pairs {fᵢ, tᵢ} may be presented to a classification algorithm at step 612 for training and normal operation. The classification algorithm may one from the algorithms described above (for example, an Artificial Neural Network) or other type of machine learning algorithm.

The exemplary algorithm presented in FIG. 6 may also be adjusted for real-time recognition of food intake that follows the same or similar sequence of processing steps. The major difference is that in real-time processing a single epoch representing the sensor signals should classified either as food intake or no food intake. Therefore, thresholds T₁ and T₂, the type of signal features to be used in classification, type and parameters of the classification algorithms as well as training of the classification algorithm have be established before use in real time.

Real time recognition of food intake enables novel, previously not possible interventions for corrections of unhealthy ingestive behaviors, such as behaviors leading to weight gain (snacking, night eating, weekend and holiday overeating) and behaviors exhibited in eating disorders such as self-limiting of food intake in cachexia and anorexia nervosa, binging and purging in bulimia. Feedback may be rpovided in real time, during the progression (or lack thereof) of an ingestive event. For example, based on the sensor signals, the amount of food that has been consumed may be calculated and a user may be warned when their food consumption for that meal or for the day has reached an optimal amount. In one embodiment, an audible or visual notification may be provided on a smart phone. In another embodiment, the feedback may be provided on actuator 214 of FIG. 2, such a wearable display or acoustical actuator (speaker/headphone/vibrator). As a result, users may easily track their food intake for the day. Other individuals may be notified that their food intake throughout the day has not been high enough, indicating they should eat more to gain weight. The wearable food monitoring system therefore has a wide application to individuals trying to maintain, gain, or lose weight.

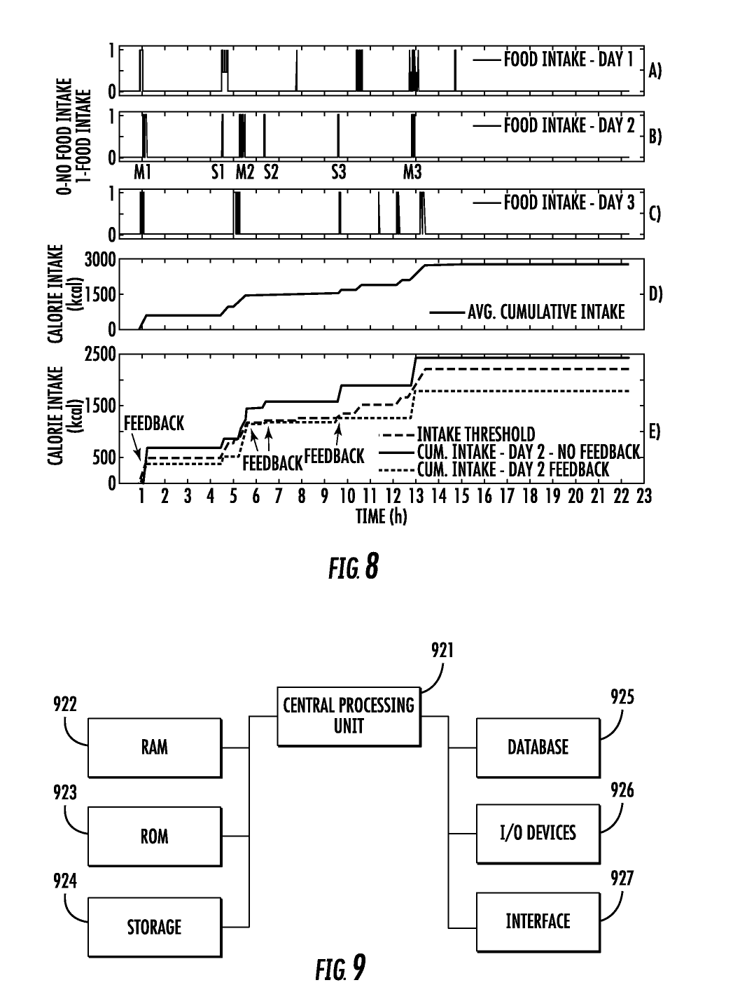

One exemplary algorithm for moderation of excessive food intake is shown on FIG. 7. Total energy intake of an individual during a day can be expressed as: EI=∫ D̅*M(t)dt, where D̅ is the average energy density of that individual's diet, M(t) is mass of intake over time and t is time (0≦t<24 h). The mass of intake over time can be estimated by the system as a linear function of number of food intake epochs N_{E}, number of chews N_{CH}, and number of hand-to-mouth gestures N_{HTM}.: M(t)=a₁N_{E}(t)+a₂N_{CH}(t)+a₃N_{HTM}(t)+ . . . +b, where a₁ . . . a_{N} are weight coefficients for each of the contributing factors and b is the intercept. Thus, to reduce someone's energy intake by a factor r<1 without a change in diet composition, it is sufficient to produce feedback that will result in a proportional reduction of eating time, number of chews, and number of hand gestures.

rEI=∫ D̅*(a₁rN_{E}(t)+a₂rN_{CH}(t)+a₃rN_{HTM}(t)+ . . . +rb)dt.

Metrics of ingestive behavior measured by the system may be functions of time (that is, behavioral patterns of ingestion) which means that the feedback will also be a function of time and will proportionally reduce EI from the eating episodes during the day. The reduction factor r may be set at a sufficiently comfortable level to avoid the feeling of hunger or dissatisfaction, for example, by reducing daily caloric intake by 10%-20%. The individual behavioral patterns can be learned by statistical modeling techniques as described next.

Individual behavioral patterns of ingestion can be extracted from the metrics computed from the food monitor data, such as various combinations of: number of swallows, swallowing frequency, relative increase in swallowing frequency in relation to baseline, number of chews, chewing rate, intensity of chewing, number of hand gestures, hand gesture rate and timing of hand gestures, number of detected food intake epochs and so on. In one exemplary implementation, Gaussian kernel smoothing may be used to obtain non-parametric probability density estimates (PDEs) for time distribution of number of chews N_{CH}(t), number of hand-to-mouth gestures N_{HTM}(t), number of food intake epochs N_{E}(t) over 24 hours using a history of ingestion over several days (step 2 on FIG. 7). Next, c̅E̅I̅(t)—an estimate of typical cumulative EI at time t will be derived using smoothed PDEs and modeling equations that use metrics (such as number of chews, etc) and/or wearable camera images to estimate nutrient and caloric intake. The estimate of c̅E̅I̅(t) will be computed following several days of observation, stored in a database (step 3 on FIG. 7) and used by the feedback algorithm. This estimate represents typical daily patterns of ingestion. The estimate can be recomputed periodically to account for changes in ingestive behavior over time.

The feedback algorithm will generate actionable feedback if the current meal/snack is approaching or is exceeding desired energy intake First, every time t when food intake is detected, an estimate of actual cumulative energy intake since the beginning of the day a̅E̅I̅(t) may be updated from real-time system data (step 4 on FIG. 7). When food intake is detected after at least 15 minutes of no intake it will be considered a start of a new eating episode and value of a̅E̅I̅(start) will be recorded. Second, a desired cumulative intake at time t will be computed from learned behavioral patterns and a desired reduction coefficient r (where r<1) specified by the researcher as d̅E̅I̅(t)=r* c̅E̅I̅(t). Third, a relative difference between actual and desired cumulative EI ΔEI(t)=( a̅E̅I̅(t)− a̅E̅I̅(start))/( d̅E̅I̅(t)− a̅E̅I̅(start)) will be used to evaluate user's progress toward allowed energy intake and generate feedback messages that will be sent to, for example, the user's phone.

FIG. 8 illustrates the operation of learning and biofeedback algorithms. Graphs a)-c) show detected food intake for three days of observation (only N_{E}(t) is shown). Graph d) shows learned average cumulative intake c̅E̅I̅(t). Graph e) shows desired intake curve d̅E̅I̅(t) as dashed line (r=0.8, reduction in intake of 20%), original intake for day 2 as solid line and intake after receiving feedback as dotted line. In this example, the algorithm delivers feedback in 4 out 6 eating episodes (only “stop eating” is shown). Meal 1 (M1) is reduced in size. Snack 1 (S1) is allowed as is. M2, S2 and S3 are reduced in size. M3 is allowed as is. Assuming the initial cumulative energy intake of 2400 kcal, the total estimated reduction after feedback is 500 kcal (from 2400 to 1900 kcal). In practical terms, the algorithms allow a certain amount of energy intake for each eating episode. For example, by learning typical ingestive patterns c̅E̅I̅(t), we know that a person usually consumes 1250 kcal in all eating episodes by the end of lunch time. If we set a reduction goal of 80% (r=0.8) then the target is consuming no more than 1000 kcal by the end of lunch time d̅E̅I̅(t). If this person had a 400 kcal breakfast and no snacks, then the algorithm will estimate the size of energy intake allowed for lunch as 600 kcal and feedback will be provided as the user is approaching the allowed energy intake. If the previous intake was 450 kcal, then the allowed energy intake will be estimated as 550 kcal and so on.

Various feedback messages may be used depending on the energy intake levels compared to desired levels. For example, the following feedback messages may be generated on a smart phone or wearable acoustical or tactile actuator. At ΔEI(t)=0.5 (actual intake for an eating episode is at 50% of allowed EI)—one short beeps. At ΔEI(t)=0.75—two short beeps (louder and higher tone). At ΔEI(t)=0.9—three short beeps and vibration. At ΔEI(t)=1.0—stop eating tune, vibration and screen message until snooze. At every 0.1 increase (1.1, 1.2, etc.)—stop eating tune, vibration and screen message until snooze. In general, the feedback may be provided as audio/tactile/visual alerts on a smart phone and/or wearable display, acoustical or tactile actuator indicating the action to be taken.

In other exemplary implementation, real-time feedback may be provided about rate of ingestion with goal either to slow down or to speed up the rate. The rate of ingestion may be characterized either as swallowing rate, chewing rate, hand gesture rate or a combination of these metrics. Real-time feedback may be provided during the meal to keep the ingestion rate at an optimal point for achieving satiety and reducing cumulative intake. The rate moderation feedback may be combined with the quantity moderation feedback.

In other exemplary implementation, real time feedback about calories being eaten may be provided through automatic processing of food imagery captured by food monitor's camera. Specific food items being eaten may be identified and portion size and nutrition information estimated from imagery. The feedback may be delivered as the number of calories presented on a wearable display in the field of view. The calorie estimates may overlay the food images. The recommended foods from available selection and recommended portion sizes may also be displayed over the captured food imagery. The image-based caloric intake feedback may be combined with rate moderation feedback and/or with the quantity moderation feedback based on sensor metrics.

FIG. 9 illustrates an exemplary processor-based computer system, on which the disclosed methods and processes may be implemented. The overall system may involve multiple sensors communicating wirelessly. The computer may include one or more hardware and/or software components configured to collect, monitor, store, analyze, evaluate, distribute, report, process, record, and/or sort information in the disclosed embodiments. For example, a controller may include one or more hardware components such as, for example, a central processing unit (CPU) 921, a random access memory (RAM) module 922, a read-only memory (ROM) module 923, a storage 924, a database 925, one or more input/output (I/O) devices 926, and an interface 927. Alternatively and/or additionally, controller 920 may include one or more software components such as, for example, a computer-readable medium including computer-executable instructions for performing a method associated with the exemplary embodiments. It is contemplated that one or more of the hardware components listed above may be implemented using software. For example, storage 924 may include a software partition associated with one or more other hardware components. The controller may include additional, fewer, and/or different components than those listed above. It is understood that the components listed above are exemplary only and not intended to be limiting.

CPU 921 may include one or more processors, each configured to execute instructions and process data to perform one or more functions associated with a controller. CPU 921 may be communicatively coupled to RAM 922, ROM 923, storage 924, database 925, I/O devices 926, and interface 927. CPU 921 may be configured to execute sequences of computer program instructions to perform various processes. The computer program instructions may be loaded into RAM 922 for execution by CPU 921.

RAM 922 and ROM 923 may each include one or more devices for storing information associated with operation of CPU 921. For example, ROM 923 may include a memory device configured to access and store information associated with controller 920, including information for identifying, initializing, and monitoring the operation of one or more components and subsystems. RAM 922 may include a memory device for storing data associated with one or more operations of CPU 921. For example, ROM 923 may load instructions into RAM 922 for execution by CPU 921.

Storage 924 may include any type of mass storage device configured to store information that CPU 921 may need to perform processes consistent with the disclosed embodiments. For example, storage 924 may include one or more magnetic and/or optical disk devices, such as hard drives, CD-ROMs, DVD-ROMs, or any other type of mass media device.

Database 925 may include one or more software and/or hardware components that cooperate to store, organize, sort, filter, and/or arrange data used by controller 920 and/or CPU 921. For example, database 925 the computations of signals from the various system sensors and a running count of calories consumed as estimated based on the food consumption. It is contemplated that database 1525 may store additional and/or different information than that listed above.

I/O devices 926 may include one or more components configured to communicate information with a user associated with controller 920. For example, I/O devices may include a console with an integrated keyboard and mouse to allow a user to input parameters or food intake. I/O devices 926 may also include a display including a graphical user interface (GUI) for outputting information on a monitor. I/O devices 926 may also include peripheral devices such as, for example, a printer for printing information associated with controller 920, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 927 may include one or more components configured to transmit and receive data via a communication network, such as the Internet, a local area network, a workstation peer-to-peer network, a direct link network, a wireless network, or any other suitable communication platform. For example, interface 927 may include one or more modulators, demodulators, multiplexers, demultiplexers, network communication devices, wireless devices, antennas, modems, and any other type of device configured to enable data communication via a communication network.

Any combination of one or more computer readable medium(s) may be utilized. The computer readable medium may be a computer readable signal medium or a computer readable storage medium. A computer readable storage medium may be, for example, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. Program code embodied on a computer readable medium may be transmitted using any appropriate medium, including but not limited to wireless, wireline, optical fiber cable, RF, etc., or any suitable combination of the foregoing.

Computer program code for may be written in any combination of one or more programming languages, including an object oriented programming language such as Java, Smalltalk, C++, or the like, and conventional procedural programming languages, such as the “C” programming language or similar programming languages. The program code may execute entirely on the computing unit.

It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

1. A system for monitoring food intake, comprising:
a jaw sensor configured to detect jaw motion;
an inertial measurement unit configured to detect body motion;
a hand gesture sensor configured to detect a hand motion; and
a central processing unit configured to determine whether the jaw motion, the body motion, and/or the hand motion are associated with food intake.

2. The system of claim 1, further including a camera for taking images of food.

3. The system of claim 1, wherein the central processing unit monitors food intake without input from an individual.

4. The system of claim 1, wherein the central processing unit is connected to one or more of the jaw sensor, the inertial measurement unit, and the hand gesture sensor using a wireless connection.

5. The system of claim 1, wherein the central processing unit uses machine learning techniques to earn food intake patterns.

6. The system of claim 1, wherein the jaw sensor is at least one of a non-contact sensor and a strain sensor.

7. The system of claim 1, further including an acoustic sensor for measuring sounds associated with food intake.

8. The system of claim 1, further including a display configured to display a notification to a user of the amount of food intake the individual has consumed over a given period of time.

9. A method for monitoring food intake, comprising:
measuring jaw motion;
measuring body motion;
measuring hand motion; and
determining whether the jaw motion, the body motion, and/or the hand motion are associated with food intake.

10. The method of claim 9, further including taking images of food.

11. The method of claim 9, further including using machine learning techniques to learn food intake patterns.

12. The method of claim 9, wherein at least one of a non-contact sensor and a strain sensor measures the jaw motion.

13. The method of claim 9, further including measuring sounds associated with food intake.

14. The method of claim 9, further including notifying a user of the amount of food intake the individual has consumed over a given period of time.

15. A computer-readable medium comprising instruction which, when executed by a processor and a memory, perform a method for monitoring food intake, comprising:
measuring jaw motion;
measuring body motion;
measuring hand motion; and
determining whether the jaw motion, the body motion, and/or the hand motion are associated with food intake.

16. The computer-readable medium of claim 15, wherein the method instruction further includes taking images of food.

17. The computer-readable medium of claim 15, wherein the method instruction further includes using machine learning techniques to learn food intake patterns.

18. The computer-readable medium of claim 15, wherein at least one of a non-contact sensor and a strain sensor measures the jaw motion.

19. The computer-readable medium of claim 15, wherein the method instruction further includes measuring sounds associated with food intake.

20. The computer-readable medium of claim 15, wherein the method instruction further includes notifying a user of the amount of food intake the individual has consumed over a given period of time.
